(19) United States
(12) Patent Application Publication
(10) Pub. No.: US 2008/0045854 A1
(43) Pub. Date: Feb. 21, 2008

(54) HUMAN BODY IMPEDANCE MEASUREMENT DEVICE AND ITS APPLICATION

(76) Inventor: Pan Weichao, Guangdong (CN)

Correspondence Address: MOORE & VAN ALLEN PLLC, P.O. BOX 13706, Research Triangle Park, NC, 27709, US

(21) Appl. No.: 11/773,683

(22) Filed: Jul. 5, 2007

(30) Foreign Application Priority Data
Aug. 16, 2006 (CN) 200610037132.7

(51) Int. Cl. A61B 5/053

(52) U.S. Cl. 600/547

(57) ABSTRACT

A device for measuring human body impedance, comprising a pair of input electrodes and a pair of output electrodes, a constant-current source, a voltage measurement unit and an operation processing unit The voltage measurement unit measures the voltage generate by the current from the constant-current source passing through the human body, and the operation processing unit calculates the human body impedance according to correction parameters. When in use, the comparison with the measurement of the standard resistor and the switching of electronic analog switches is omitted, which greatly reduces the period of measurement lowers the cost of the product simplifies the circuit strengthens the anti-interference capability of the circuit, and cuts down the power consumption of the circuit A human body fat meter using the device for measuring the human body impedance is also disclosed, which quickly and precisely converts the impedance into fat content of the human body.

### STATEMENT OF RELATED APPLICATION

The present application claims priority of the Chinese Patent Application No. 200610037132.7, entitled “a human body impedance measurement device and a fat meter using the same”, filed on Aug. 16, 2006, which is incorporated herein by reference in its entirety.

### FIELD OF THE INVENTION

The present invention relates to a device for measuring the human body impedance and a fat meter using the device.

### BACKGROUND OF THE INVENTION

It has long been a subject in the academic field to measure fat content in a scientific way. At present, the commonly accepted methods for measuring the human body fat are mainly classified into following types: a) a method of absorbing dual energy X-rays which is to measure fat content according to that different parts of the human body can absorb X-rays to different extent; b) a method of measurement by near infrared rays which is to measure fat content by having the near infrared light with low energy emitted from the optical fibers irradiate the biceps; c) a method of measurement by a skin gauge which is to measure the thickness of subcutaneous fat at different parts of the human body using a special gauge and then calculate the fat content in the formula; d) an underwater weighing method which is to measure fat content according to the principle that the specific gravity of human body fat is smaller than that of water.

Unfortunately, since above-mentioned methods have, more or less, various problems such as high requirement of technique, inconvenient operation, complex calculation, they are only suitable to the professional institutes such as medical treatment institutes, difficult to be applied by ordinary families.

With the development of technology, there is proposed a method of measuring the bio-impedance which is to measure fat content by having the low-voltage current passing through the human body according to that different tissues of the human body have different impedance to the current. This method is simple and convenient for easily measuring fat and water content, enabling people to know their own figure index.

As shown in FIG. 1, the basic principle of the existing method of measuring human body fat by measuring the bio-impedance is: applying a constant alternating sine wave current generated by a constant-current source to the human body and a standard resistor; detecting the voltage drop generated when the current passing through the human body by applying a voltage detector, comparing said voltage drop with that generated when the current passing through the standard resistor to obtain the human body impedance, and finally calculating the fat content of the human body through a specified calculation method. In particular, the output ends of the constant-current source is connected in series with two standard resistors (wherein the value of one resistance is about 300Ω, and that of the other is about 500Ω) and the human body to be measured, said standard resistors and the human body to be measured are provided with contacts on both ends, the input end of a voltage detector is connected with an analog switch, and the other contact of the analog switch is respectively connected with the contacts of the standard resistors and that of the human body to be measured, so as to respectively obtain the voltage of the standard resistors and that of the human body to be measured. According to Ohm's law, the relation between voltage and resistance is the linear relation Rₓ=(Uₓ□U₀)/I₀. With reference to FIG. 2, the initial voltage value U₀ and the value I₀ of the current from the constant-current source are calculated according to the resistance of the standard resistor and the corresponding voltage value, and then the human body impedance Rₓ is calculated according to the voltage Uₓ of the human body to be measured.

The existing products manufactured according to above-mentioned method have following disadvantages: (1) each product is equipped with more than two correction resistors and relatively large amount of electronic analog switches, with complex circuit, high cost and poor anti-interference; (2) a plurality of correction resistors have to be measured each time when using the product, so the period for the measurement is prolonged; (3) since the correction resistor is connected in series with the human body, which cause relatively high total loop resistance, the power supply voltage is required to be relatively high, which is not favorable for supplying power using batteries, or the current value must be reduced, but the precision of measurement will be finally affected and clipping distortion is easily to occur, affecting the measurement result. In addition, the existing method of measuring human body fat does not verify the contact situation between both feet during measurement, so the measurement result will be incorrect when the contact between both feet and the measurement electrodes is poor.

### SUMMARY OF THE INVENTION

One aspect of embodiments of the present invention is to provide a device for measuring the human body impedance, omitting the comparison with a measurement standard resistor, and the switching of electronic analog switches, which greatly reduces the period of the measurement, lowers the cost of the product, simplifies the circuit, strengthens the anti-interference capability of the circuit and cuts down the power consumption of the circuit.

Another aspect of embodiments of the present invention is to provide a human body fat meter, which quickly and precisely converts the impedance into the fat content of the human body after the impedance is measured by the device for measuring the human body impedance.

To achieve above-mentioned aspects, the technical solutions adopted by the invention are as follows.

According to the first aspect of embodiments of the present invention, there is provided a device for measuring the human body impedance, comprising: a pair of input electrodes (1, 1′) in contact with the human body and introducing an alternating excitation current into the human body; a pair of output electrodes (2, 2′) in contact with the human body and detecting a voltage drop generated in the human body by the excitation current; a constant-current source (3) for supplying the human body with the alternating excitation current via the electrodes (1, 1′); a voltage measurement unit (4) for measuring the voltage drop in the human body generated by the excitation current; and an operation processing unit (5) that stores correction parameters and conducts operation on the correction parameters and the voltage measured by the voltage measurement unit (4) so as to calculate the human body impedance; wherein the calculation of the human body impedance is carried out as follows: the voltage measurement unit (4) measures the voltage generated by the current from the constant-current source (3) passing through the human body, and the operation processing unit (5) calculates the human body impedance based on the voltage and the correction parameters.

The correction parameters at least comprise an output current of the constant-current source and a zero-point voltage, also comprise temperature compensation coefficients of the voltage and current, which means the operation processing unit (5) makes temperature compensation for the calculated the human body impedance according to the environmental temperature, and further comprise a drive capacity compensation coefficient of the constant-current source, for compensating the error resulted from variation of the output current of the constant-current source (3) due to the variation in load resistance.

The operation processing unit (5) judges whether the contact between the human body and the input and output electrodes (1, 1′, 2, 2′) is perfect according to the voltage range measured on the input electrode, and gives a corresponding indication.

The voltage measure unit (4) comprises a differential amplifier (41), a rectifier (42) and an A/D converter (43). The output electrodes (2, 2′) are respectively connected with the non-inverting and inverting inputs of the differential amplifier (41). An alternating voltage signal with a certain level is obtained at the output of the differential amplifier (41), and the alternating voltage signal is inputted into the rectifier (42) so as to obtain a DC voltage signal with a corresponding level value; the DC voltage signal is transmitted to the A/D converter (43), which converts the corresponding analog voltage signal into a digital signal; and then the operation unit (5) calculates the human body impedance according to the correction parameters. An analog switch (6) is connected between the output electrode (2, 2′) and the differential amplifier (41), terminals on one side of which are connected with the non-inverting and inverting inputs of the differential amplifier (41) respectively and terminals on the other side are connected with the input electrodes (1, 1′) or the output electrodes (2, 2′); and the control terminal of the switch (6) is connected with the operation unit (5).

According to the second aspect of embodiments of the present invention, a human body fat meter incorporating the device for measuring the human body impedance according to the first aspect of the present invention is provided, comprising: a pair of input electrodes (1, 1′) in contact with a human body and introducing an alternating excitation current into the human body; a pair of output electrodes (2, 2′) in contact with the human body and detecting a voltage drop generated in the human body by the excitation current; a constant-current source (3) for supplying the human body with the alternating excitation current via the input electrodes (1, 1′); a voltage measurement unit (4) for measuring the voltage drop generated in the human body by the excitation current; and an operation processing unit (5) that stores the correction parameters and conducts operation on the correction parameters and the voltage measured by the voltage measurement unit (4) so as to calculate the human body impedance; wherein the calculation of the human body fat content is carried out as follows: the voltage measurement unit (4) measures the voltage generated by the current from the constant-current source (3) passing through human body, and the operation processing unit (5) calculates the human body impedance based on the voltage and the correction parameters, and then calculates fat content of the human body according to the corresponding relation between the human body impedance, human body parameters and the fat content, which is finally displayed by a display unit (7) in communication with the operation processing unit (5).

Optionally, the human body fat meter according to the second aspect of embodiments of the present invention is further provided with a weight measurement unit (8), which is connected with the operation processing unit (5).

The advantageous effects of embodiments of the present invention are: with regard to the drawbacks of correction methods using standard resistors in conventional solutions, embodiments of the present invention adopt a correction method that stores the correction parameters in advance, only necessary to detect the voltage generated by the current passing through a human body when in use, to read the correction parameter from the memory, and then to calculate the human body impedance according to the linear relation between the human body impedance and the voltage. Upon the method in embodiments of the present invention, it is to omit the comparison with the measurement of the standard resistor, and the switching of electronic analog switches which greatly reduces the period of measurement, lows down the cost of the product, simplifies the circuit, strengthens the anti-interference capability of the circuit and cuts down power consumption of the circuit. Moreover, various influence factors affecting the measurement of the human body impedance are sufficiently considered when the device of embodiments according to the invention is provided, including variation in environmental temperature and deficiency of drive capacity of the constant-current source, and the corresponding influence parameters are used to correct the calculation of the human body impedance so as to make the result more precise. In addition, the device of the invention also judges whether or not the contact is perfect or the user is wearing stockings by measuring the voltage on the input electrodes. The human body fat meter quickly and precisely converts the impedance into the fat content of the human body after the impedance is measured by above-mentioned device of measuring the human body impedance.

### BRIEF DESCRIPTION OF THE DRAWINGS

The present invention is further illustrated through following embodiments and appended drawings:

FIG. 1 is a structural diagram of a device for measuring the human body impedance in prior art;

FIG. 2 is a graph showing the relation between the voltage and resistance;

FIG. 3 is a structural block diagram of a device for measuring the human body impedance according to an example embodiment of the present invention;

FIG. 4 is a connection diagram of calibrating zero-point voltage by measuring the human body impedance according to an example embodiment of the present invention;

FIG. 5 is a connection diagram of calibrating the output current from the constant-current source by measuring the human body impedance according to an example embodiment of the present invention;

FIG. 6 is a graph showing the relation between the output current from the constant-current source and the load resistance;

FIG. 7 is a connection diagram of the drive capacity compensation coefficient of the constant-current source calibrated by measuring the human body impedance according to an example embodiment of the present invention;

FIG. 8 is a graph showing the relation between the voltage and resistance corrected by the temperature and the constant-current source drive capacity;

FIG. 9 is a structural block diagram of the human body fat meter according to an example embodiment of the present invention.

### DETAILED DESCRIPTION OF THE INVENTION

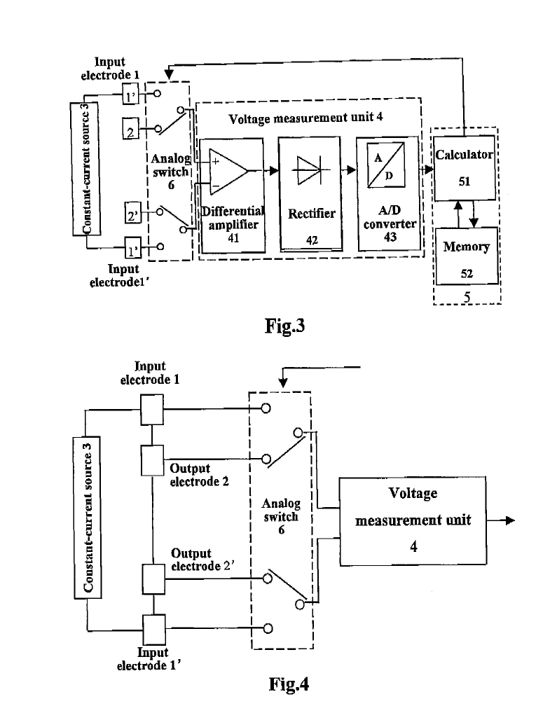

With reference to FIG. 3, a device for measuring the human body impedance is shown, which comprises a pair of input electrodes 1, 1′ in contact with the human body and introducing an excitation current into the human body; a pair of output electrodes 2, 2′ in contact with the human body and detecting the voltage drop generated in the human body by the excitation current; a constant-current source 3 for supplying the human body with constant alternating excitation current through the input electrodes 1, 1′; a voltage measurement unit 4 for measuring the voltage drop generated in the human body by the excitation current; and an operation processing unit 5 that stores the correction parameters and operates processing on the correction parameters and the voltage measured by the voltage measurement unit 4 so as to calculate the human body impedance. The calibrating calculation of the human body impedance is carried out as follows: the voltage measurement unit 4 measures the voltage generated by the current from the constant-current source 3 passing through the human body, and the operation processing unit 5 calculates the human body impedance Rₓ according to the correction parameters.

The voltage measure unit 4 comprises a differential amplifier 41, a rectifier 42 and an A/D converter 43. The output electrodes 2, 2′ are respectively connected with the non-inverting and inverting input ends of the differential amplifier 41. An alternating voltage signal with a certain level is obtained at the output of the differential amplifier 41 and inputted into the rectifier 42 so as to obtain a DC voltage signal with a corresponding level value; the DC voltage signal is transmitted to the A/D converter 43, which converts the corresponding analog voltage signal into a digital signal; and then the operation processing unit 5 calculates the human body impedance Rₓ based on the digital signal and the correction parameters.

The operation processing unit 5 comprises a calculator 51 and a memory 52. The calculator 51 is connected with the output end of the voltage measurement unit 4 to obtain the digital signal corresponding to the voltage signal. The memory 52 is connected with the calculator 51, which stores the correction parameters therein, comprising a zero-point voltage and the value of the output current from the constant-current source 3. The calculator 51, according to the linear relation between the voltage signal and the human body impedance, works out the human body impedance Rₓ according to the zero-point voltage U₀ and the current value 10 of the constant-current source 3.

The operation process and principles of the device for measuring the human body impedance is described in detail.

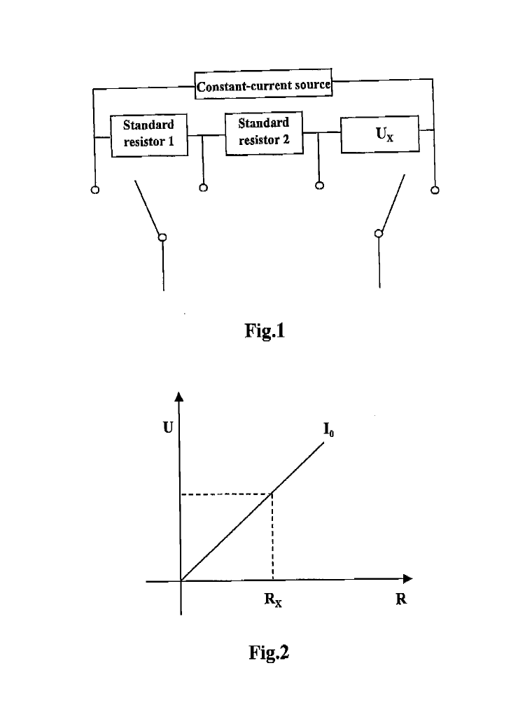

A□ Parameter calibration. According to the linear relation between the voltage and the resistance Rₓ□(Uₓ−U₀)/I₀, with reference to FIG. 2, the parametric zero-point voltage U₀ and the value I₀ of the output current from the constant-current source 3 need to be calibrated. First, with reference to FIG. 4, when the four electrodes, namely the input electrodes 1, 1′ and the output electrodes 2, 2′ are short-circuited, the voltage measured by the voltage measurement unit 4 is the zero-point voltage U₀ which is stored by the operation processing unit 5 into the memory 52 at the specified address. Then, with reference to FIG. 5, when the input electrodes 1, 1′ and input electrodes 2, 2′ are respectively short-circuited, and a standard precision resistor R₀ with a resistance equal to 1000Ω is connected between the input electrode 1 and the output electrode 2, the voltage measured by the voltage measurement unit 4 is U_{R0}. According to Ohm's law namely I₀□(U_{R0}−U₀)/R₀, the value I₀ of the current from the constant-current source 3 is calculated, which is stored by the operation processing unit 5 into the memory 52 at the specified address.

B. Measurement of the human body impedance. When measurement is processed, both feet of the human body are in contact with the input electrodes 1, 1′ and the output electrodes 2, 2′. The generated current in the constant-current source is inputted into the human body via the input electrodes 1, 1′, and then the voltage drop is generated between the output electrodes 2, 2′ when the current passing through the human body. The voltage measurement unit (4) measures the voltage Uₓ across the output electrodes 2, 2′, and sends the voltage to the operation processing unit 5; meanwhile the operation processing unit 5 reads the correction parameters I₀ and U₀ from the memory 52, and then calculates the human body impedance Rₓ through the formula Rₓ□(Uₓ−U₀)/I₀.

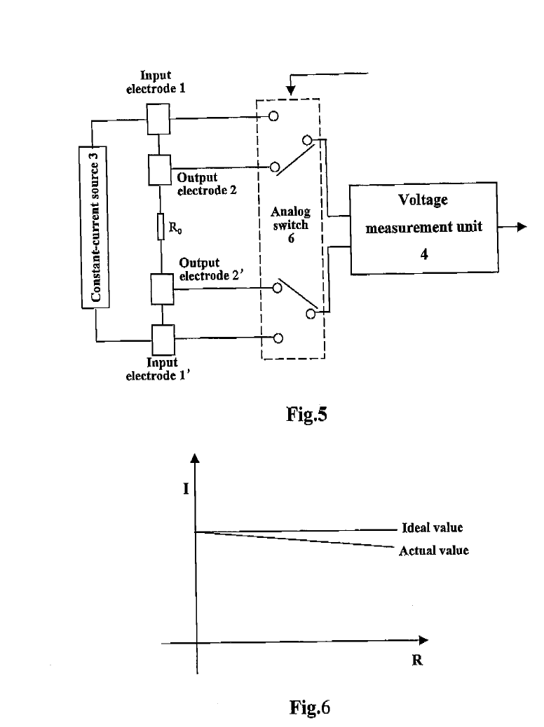

The measured correction parameters are written into the memory 52 by the manufacturer when the devices are consigned. With reference to FIG. 6, in case the temperature upon the users using the device is different from that when the correction parameters are written by the manufacturer, the electrical parameters of the differential amplifier 41, rectifier 42, A/D converter 43 and the like in the voltage measurement unit 4 will drift due to the change of the temperature, so that the zero-point voltage U₀ and the value I₀ of the current from the constant-current source 3 will change accordingly.

(1) Temperature coefficient correction, including voltage temperature coefficient and current temperature coefficient. Assuming that the calibrated temperature set by the manufacturer is T₀ and the temperature upon users using the device is T₁, the zero-point voltage parameter is U₁ and the output current of the constant-current source 3 is I₁ when the device in use, with following:

U₁=U₀[1+(T₁−T₀)K₁],

I₁=I₀[1+(T₁−T₀)K₂];

wherein K₁, K₂ are voltage temperature coefficient and current temperature coefficient respectively.

(2) Compensation for the drive capacity of the constant-current source. Ideally, for different loads, the drive capacity of the constant-current source 3 should be consistent, and the voltage generated on the load resistor is linearly related to the resistance. However, in actual measurement, with variation in the load resistance, the output voltage of the constant-current source 3 also varies, and the actual output current from the constant-current source 3 will make a change. In general, the output current from the constant-current source 3 decreases when the load resistance increases, as shown in FIG. 6. The relation between the actual output current and output voltage of the constant-current source can be calculated by measuring the load resistance and the voltage generated on the load resistor, so as to make compensation for the actual output current from the constant-current source 3.

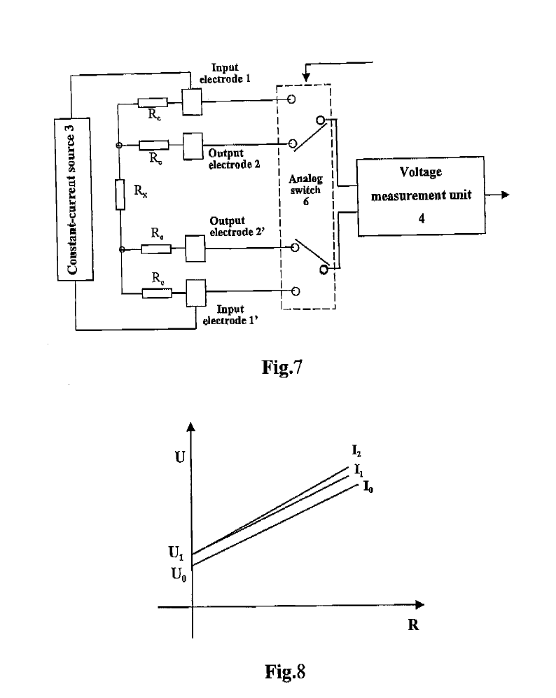

When measuring the human body impedance Rₓ, the contact resistance R_{c} between the human body and the electrode varies due to various factors, such as skin wetting degree, size of contact area, thickness of keratoderma. The Kelvin's bridge adopted by the present invention may decrease the influence of the contact resistance R_{c} on the measurement result. However, the increase of the contact resistance R_{c} may cause variation in the output current from the constant-current source, thereby to some extent affecting measurement precision of the human body impedance Rₓ. To make the measurement more precise, the present invention utilizes the technique of drive capacity correction, as shown in FIGS. 7 and 8. Upon measurement, the voltage measure unit 4 is connected with the input electrodes 1, 1′, to detect the total voltage drop U_{c} generated by the current from the constant-current source 3 passing through the contact resistance and human body. Assuming that the value of the actual output current from the constant-current source 3 is I₂ under a certain load, and the temperature influence factor is taken into consideration, the following is provided:

I₂=I₁[1+(U_{c}−U₁)K₃],

wherein K₃ is the current drive capacity coefficient. Finally, the calculation formula for the human body impedance Rₓ is Rₓ□(Uₓ−U₁)/I₂, and the human body impedance Rₓ can be calculated by substituting the above U₁, U_{c}, I₁ and I₂ into the formula. The voltage temperature coefficient K₁, current temperature coefficient K₂ and current drive capacity coefficient K₃ are all stored in the memory 52. When in use, the calculator 51 upon operation will accordingly use the voltage temperature coefficient K₁, current temperature coefficient K₂ and current drive capacity coefficient K₃ to make temperature correction to the linear relation between the voltage signal and the human body impedance.

Upon measurement of the human body impedance Rₓ, if the contact between the skin and the electrode is poor, e.g., in case of wearing stockings, the resistance of the whole loop is high, the theoretical output voltage of the constant-current source 3 is beyond the range of the actual voltage in operation; the output current also becomes inconstant, leading to clipping distortion, which will badly affect the measurement result. Therefore an analog switch 6 is connected between the output electrodes 2, 2′ and the differential amplifier 41. The terminals on one side of the switch 6 is respectively connected with the non-inverting and inverting input ends of the differential amplifier 41, and the terminals on the other side is connected with the input electrodes 1, 1′ or the output electrodes 2, 2′, and the control terminal of the switch 6 is connected with the operation unit 5. When in use, the analog switch 6, is in connection with the input electrodes 1, 1′ via the control of the operation unit 5, to detect the voltage U_{c} across the input electrodes 1, 1′. In case the detected voltage U_{c} is larger than the predetermined voltage Uₘₐₓ, it means that the contact resistance is too large, that is, the contact between the human body and the electrodes is poor, and then the operation processing unit 5 gives a corresponding indication. In case the detected voltage U_{c} is within a reasonable range, the analog switch 6 is in connection with the output electrodes 2, 2′ via the control of the operation processing unit 5 to detect the voltage Uₓ across the output electrodes 2, 2′, and then the operation processing unit 5 calculates the human body impedance Rₓ.

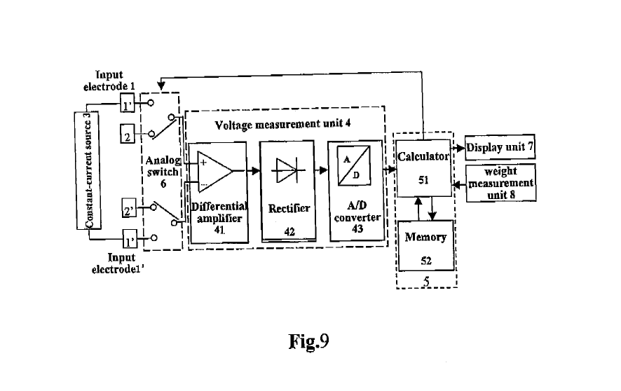

Based on the device for measuring the human body impedance operable to measure the impedance, a human body fat meter can be made according to the corresponding relation between the human body impedance Rₓ and the fat content of the human body. As shown in FIG. 9, a human body fat meter according to the present embodiment comprises a pair of input electrodes 1, 1′ in contact with the human body and introducing an alternating excitation current into the human body; a pair of output electrodes 2, 2′ in contact with the human body and detecting the voltage drop generated in the human body by the excitation current; a constant-current source 3 for supplying the human body with constant excitation current via the input electrodes 1, 1′; a voltage measurement unit 4 for measuring the voltage drop generated in the human body by the excitation current; and an operation processing unit 5 that stores the correction parameters and conducts operation on the correction parameters and the voltage measured by the voltage measurement unit 4 so as to calculate the human body impedance; wherein the calibrating calculation of the fat content of the human body impedance is carried out as follows: the voltage measurement unit 4 measures the voltage generated by the current from the constant-current source 3 passing through the human body, and the operation processing unit 5 calculates the human body impedance Rₓ based on the voltage and the correction parameters, and then calculates the fat content of the human body according to the corresponding relation between the human body impedance Rₓ, the human body parameters (stature, weight, etc.) and the fat content, which is finally displayed by the display unit 7 in communication with the operation processing unit 5. Of course, the conversion relation between the human body impedance Rₓ and the fat content involves the weight of the human body, so the human body fat meter is further provided with the weight measurement unit 8, which is connected with the operation unit 5, to obtain directly the weight parameter of the human body without requiring additional input.

In view of the drawbacks of correction methods using standard resistors in conventional solutions, embodiments of the present invention adopt a correction method that stores the correction parameters in advance, when in use, only necessary to detect the voltage generated by the current passing through the human body, read the correction parameters from the memory, and then calculate the human body impedance according to the linear relation between the human body impedance and the voltage. With embodiments of the present invention, it is to omit the comparison with the measurement of the standard resistor and the switching of electronic analog switches, which greatly reduces the period of measurement, lowers the cost of the product, simplifies the circuit, strengthens the anti-interference capability of the circuit, and cuts down the power consumption of the circuit. Various influence factors affecting the measurement of the human body impedance are also sufficiently considered in the device of the invention, including variation in environmental temperature and deficiency of the drive capacity of the constant-current source, and the corresponding influence parameters are used for correcting the calculation of the human body impedance so as to make the result more precise. In addition, the device of embodiments of the invention also judges whether or not the contact is perfect or the user is wearing stockings by measuring the voltage across the input electrodes 1, 1′. The human body fat meter quickly and precisely converts the impedance into the fat content of the human body after the impedance is measured by above-mentioned device for measuring the human body impedance.

1. A device for measuring a human body impedance, comprising:
a pair of input electrodes in contact with the human body and introducing an alternating excitation current into the human body,
a pair of output electrodes in contact with the human body and detecting a voltage drop generated in the human body by the alternating excitation current;
a constant-current source for supplying the human body with the alternating excitation current via the input electrodes;
a voltage measurement unit for measuring the voltage drop generated in the human body by the excitation current; and
an operation processing unit that stores correction parameters and conducts operation on the correction parameters and the voltage measured by the voltage measurement unit so as to calculate the human body impedance;
wherein the calculation of the human body impedance is carried out as follows: the voltage measurement unit measures the voltage generated by the current from the constant-current source passing through the human body, and the operation processing unit calculates the human body impedance based on the voltage and the correction parameters.

2. The device according to claim 1, wherein the correction parameters comprise at least an output current of the constant current source and a zero-point voltage.

3. The device according to claim 2, wherein the correction parameters further comprise temperature compensation coefficients of the voltage and the current; the operation processing unit performing temperature compensation for the calculated human body impedance according to an environmental

4. The device according to claim 2, wherein the correction parameters further comprise a drive capacity compensation coefficient of the constant-current source, for compensating for an error resulting from a variation of the output current from the constant current source due to a variation in a load resistance.

5. The device according to claim 3, wherein the correction parameters further comprise the drive capacity compensation coefficient of the constant-current source, for compensating for an error resulting from a variation in the output current from the constant-current source due to a variation in load resistance.

6. The device according to claim 1, wherein the operation processing unit judges whether or not the contact between the human body and the input and output electrodes is perfect according to a voltage range measured on the input electrodes, and gives a corresponding indication

7. The device according to claim 1, wherein the voltage measure unit comprises a differential amplifier, a rectifier and an A/D converter, the output electrodes are respectively connected with the non-inverting and inverting inputs of the differential amplifier, an alternating voltage signal with a certain level is obtained at the output of the differential amplifier, the alternating voltage signal being inputted into the rectifier so as to obtain a DC voltage signal with a corresponding level value; the DC voltage signal being transmitted to the A/D converter, which converts the corresponding analog voltage signal into a digital signal, the operation processing unit calculating the human body impedance based on the digital signal and the correction parameters.

8. The device according to claim 7, wherein an analog switch is connected between the output electrode and the differential amplifier, terminals on one side of the switch being connected with the non-inverting and inverting input ends of the differential amplifier respectively, and terminals on the other side being connected with the input electrodes or the output electrodes; and the control terminal of the switch being connected with the operation processing unit.

9. A human body fat meter, comprising:
a pair of input electrodes in contact with the human body and introducing an alternating excitation current into the human body;
a pair of output electrodes in contact with the human body and detecting a voltage drop generated in the human body by the alternating excitation current;
a constant-current source for supplying the human body with the alternating excitation current via the input electrodes;
a voltage measurement unit for measuring the voltage drop generated in the human body by the excitation current; and
an operation processing unit that stores correction parameters and conducts operation on correction parameters and the voltage measured by the voltage measurement unit to calculate the human body impedance;
wherein the calculation of a fat content of the human body includes: the voltage measurement unit measuring the voltage generated by the current from the constant-current source passing through the human body, and the operation processing unit calculating the human body impedance based on the voltage and the correction parameters, and calculating the fat content of the human body according to a corresponding relation between the human body impedance, human body parameters and the fat content, which is displayed by a display unit in communication with the operation processing unit.

10. The human body fat meter according to claim 9, further comprising a weight measurement unit connected with the operation processing unit.

11. The human body fat meter according to claim 9, wherein the correction parameters comprise at least an output current of the constant-current source and a zero-point voltage.

12. The human body fat meter according to claim 9, wherein the correction parameters further comprise temperature compensation coefficients of the voltage and the current; the operation processing unit performing temperature compensation for the calculated human body impedance according to an environmental temperature.

13. The human body fat meter according to claim 9, wherein the correction parameters further comprise a drive capacity compensation coefficient of the constant-current source, for compensating for an error resulting from a variation of the output current from the constant-current source due to a variation in a load resistance.

14. The human body fat meter according to claim 9, wherein the correction parameters further comprise a drive capacity compensation coefficient of the constant-current source, for compensating for an error resulting from a variation in the output current from the constant-current source due to a variation in load resistance.

15. The human body fat meter according to claim 9, wherein the operation processing unit judges whether or not the contact between the human body and the input and output electrodes is perfect according to a voltage range measured on the input electrodes, and gives a corresponding indication.

16. The human body fat meter according to claim 9, wherein the voltage measure unit comprises a differential amplifier a rectifier and an A/D converter, the output electrodes are respectively connected with the non-inverting and inverting inputs of the differential amplifier, an alternating voltage signal with a certain level is obtained at the output of the differential amplifier, which is inputted into the rectifier so as to obtain a DC voltage signal with a corresponding level value; the DC voltage signal being transmitted to the A/D converter, which converts a corresponding analog signal of the voltage signal into a digital signal, and the operation processing unit calculating the human body impedance based on the digital signal and the correction parameters.

17. The human body fat meter according to claim 9, wherein an analog switch is connected between the output electrode and the differential amplifier, terminals on one side of the switch being connected with the non-inverting and inverting inputs of the differential amplifier respectively, and terminals on the other side being connected with the input electrodes or the output electrodes; and the control terminal of the switch being connected with the operation processing unit
